(19) United States
(12) Patent Application Publication
(10) Pub. No.: US 2021/0298897 A1
(43) Pub. Date: Sep. 30, 2021

(54) ASSEMBLY FOR A CLOSURE DEVICE WHICH CAN BE IMPLANTED INTO THE SUPERIOR OR INFERIOR VENA CAVA OF A HUMAN BODY IN A MINIMALLY INVASIVE MANNER, AND TRICUSPID VALVE PROSTHESIS WHICH CAN BE IMPLANTED IN A MINIMALLY INVASIVE MANNER

(71) Applicant: Immanuel Albertinen Diakonie GmbH, Hamburg (DE)

(72) Inventor: Christian BUTTER, Berlin (DE)

(73) Assignee: Immanuel Albertinen Diakonie GmbH, Hamburg (DE)

(21) Appl. No.: 17/260,766

(22) PCT Filed: Jul. 16, 2019
(86) PCT No.: PCT/DE2019/100658

§ 371 Date: Jan. 15, 2021

(30) Foreign Application Priority Data
Jul. 17, 2018 (DE) 10 2018 117 292.3

(51) Int. Cl. A61F 2/24

(52) U.S. Cl. CPC A61F 2/2418; A61F 2220/0008

(57) ABSTRACT

The disclosure relates to an arrangement of a minimally-invasive implantable closing device (1) in the superior or inferior vena cava of a human body, with a valve device (6), in which closing elements (9), which in each case extend flat over a joint surface, and can be moved between a closing position, in which the closing elements (9) together close a valve opening, and an opening position, in which a flow is released through the valve opening; an anchoring device (7) that has a self-expandable anchor and is set up to anchor the valve device (6) in the area of the superior or inferior vena cava adjacent to the vein opening in the right chamber of the heart; and a flexible packing collar (8). A minimally-invasive implantable tricuspid valve prosthesis is also provided.

The invention relates to an arrangement of a minimally-invasive implantable closing device as well as a minimally-invasive implantable tricuspid valve prosthesis in the superior or inferior vena cava of a human body.

### BACKGROUND

A minimally-invasive implantable mitral or tricuspid valve prosthesis is known from the document EP 3 231 393 A1. For positioning and attaching, the valve prosthesis is securely connected to a self-expanding, broad-meshed stent, which is adapted in its shape to a three-dimensional measured geometry of a patient's left or right chamber and after implantation rests on the inside wall of the chamber.

### SUMMARY

The object of the invention is to indicate an arrangement of a minimally-invasive implantable closing device as well as a minimally-invasive implantable tricuspid valve prosthesis in the superior or inferior vena cava of a human body, which device and prosthesis can be securely located at the site of the implantation and ensure a secure opening and closing.

For a solution, an arrangement of a minimally-invasive implantable closing device in the superior or inferior vena cava of a human body is provided. The coordinate claim 16 relates to a minimally-invasive implantable tricuspid plate prosthesis. Additional configurations are the subject matter of dependent subclaims.

According to one aspect, an arrangement of a minimally-invasive implantable closing device in the superior or inferior vena cava of a human body is provided, having: a valve device, in which closing elements, which in each case extend flat over a joint surface, and can be moved between a closing position, in which the closing elements together close a valve opening, and an opening position, in which a flow is released through the valve opening; an anchoring device that has a self-expandable anchor and is set up to anchor the valve device in the area of the superior or inferior vena cava adjacent to the vein opening in the right chamber of the heart; and a flexible packing collar.

According to another aspect, a minimally-invasive implantable tricuspid valve prosthesis is provided with such an arrangement.

The proposed closing device can be securely attached at the implantation site by means of the anchoring device. The valve device with the closing elements ensures a reliable opening and closing in order to prevent or pass the flow.

Adjacent closing elements can overlap in edge-side sections at least in the closing position.

The valve device can be formed as a one-way valve.

Some or all of the closing elements can be correspondingly arranged in the closing position of a closing surface of a cone peripheral surface.

Some or all of the closing elements can be arranged in the closing position to form a closing surface that is arranged crosswise to a direction of flow.

The closing elements can be moved some distance apart to move from the closing position into the opening position by means of lifting in the direction of flow.

The closing elements can be moved by means of pivoting between the closing position and the opening position.

The closing elements can pivot around a pivoting axis, which elements are arranged in the area of a plane that is stretched from the flexible packing collar or adjacent thereto.

The pivoting axis can be formed to intersect the valve opening.

Some or all of the closing elements can be connected at least in pairs by means of holding elements.

Some or all of the closing elements can be designed as surface-rigid closing elements.

The anchoring device can have at least one of the following anchoring sections: a first anchoring section, which is covered with pericardium or a synthetic membranous plastic material, and a second anchoring section, which is free of pericardium and the synthetic membranous plastic material.

The self-expandable anchor can have at least one anchor from the following group: self-expandable spiral element and self-expandable stent.

Some or all of the closing elements can consist of a biological material. As biological material, for example, pericardium can be used.

Some or all of the closing elements can consist of a non-biological material. A usable non-biological material is, for example, plastic.

### DESCRIPTION OF EMBODIMENTS

Below, additional embodiments are explained in greater detail with reference to the figures of a drawing. In this case:

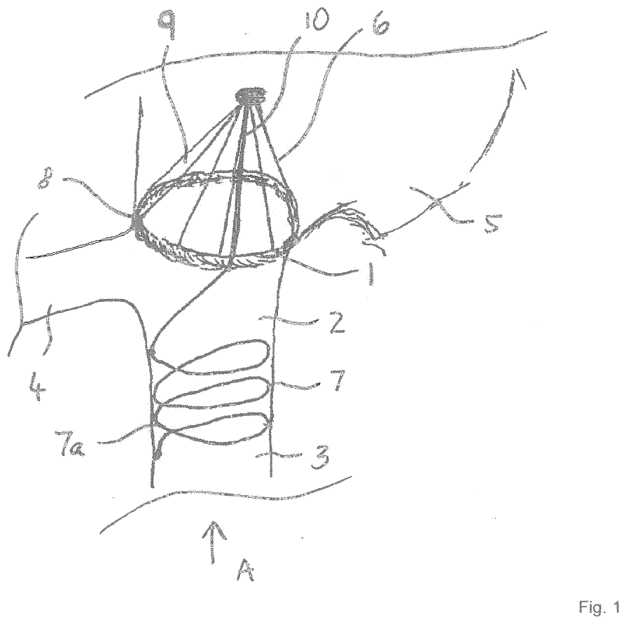

FIG. 1 shows a diagrammatic depiction of a closing device that is implanted in a vein;

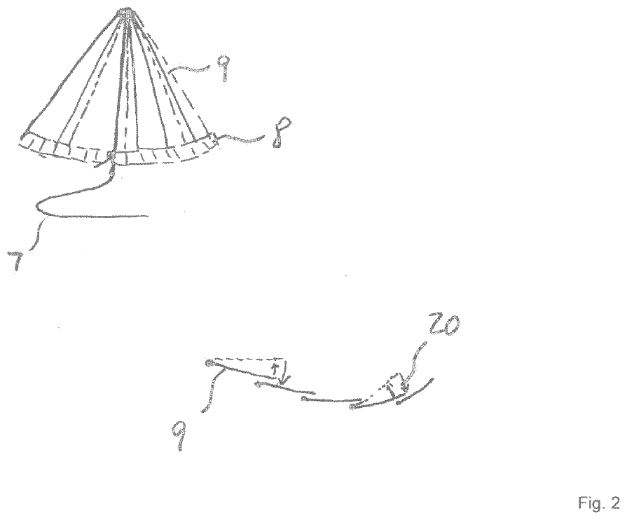

FIG. 2 shows a diagrammatic depiction of elements of the closing device of FIG. 1;

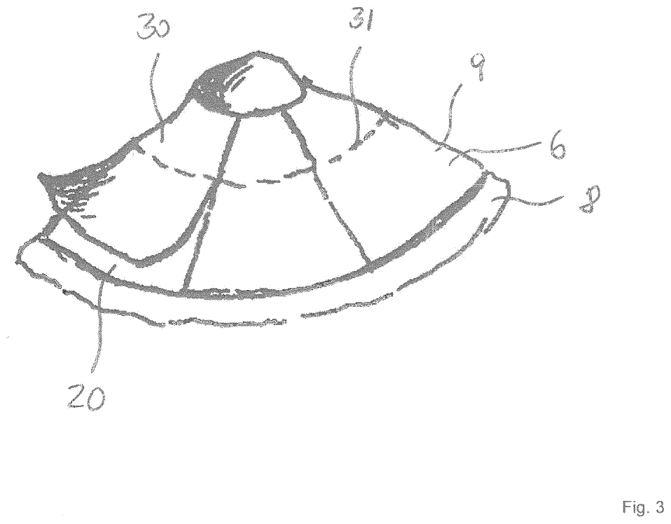

FIG. 3 shows a diagrammatic perspective depiction of elements of a closing device in a different embodiment;

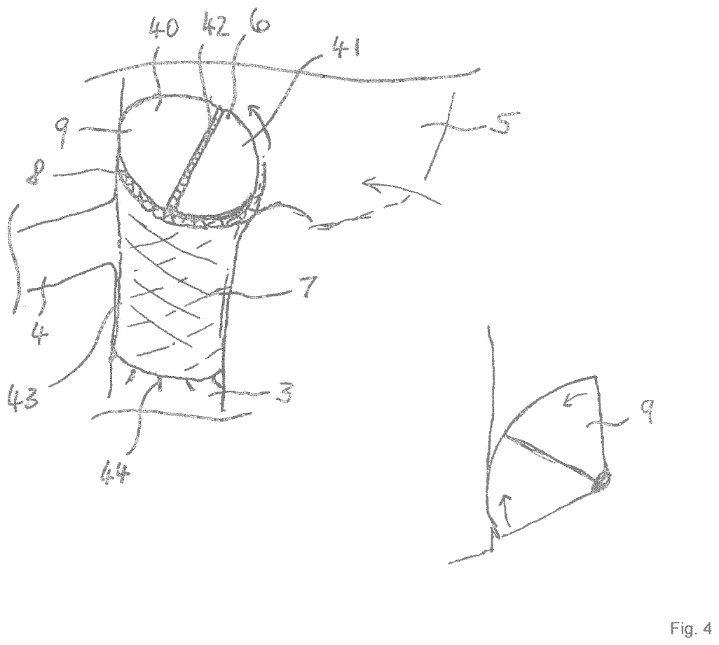

FIG. 4 shows a diagrammatic depiction of another closing device, which is implanted in the area of a vein;

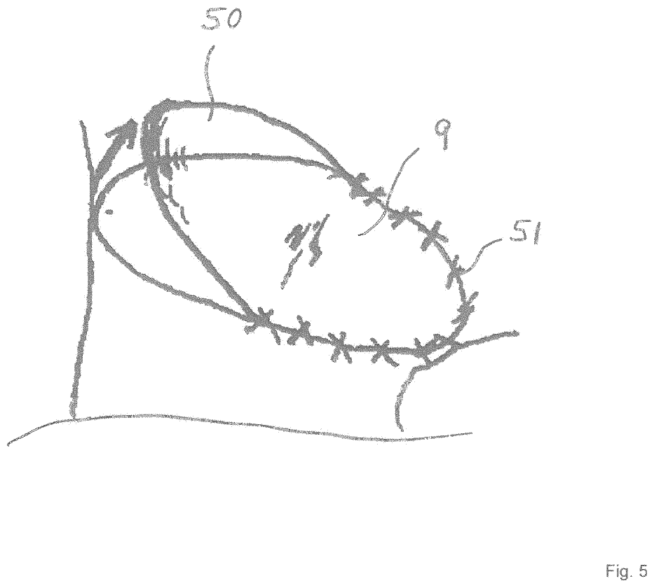

FIG. 5 shows a diagrammatic depiction of a different closing device;

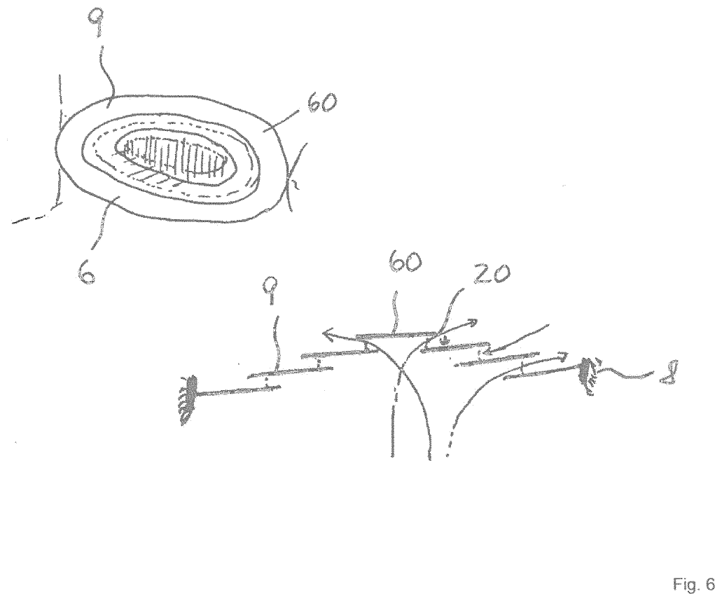

FIG. 6 shows a diagrammatic depiction of closing elements of another closing device;

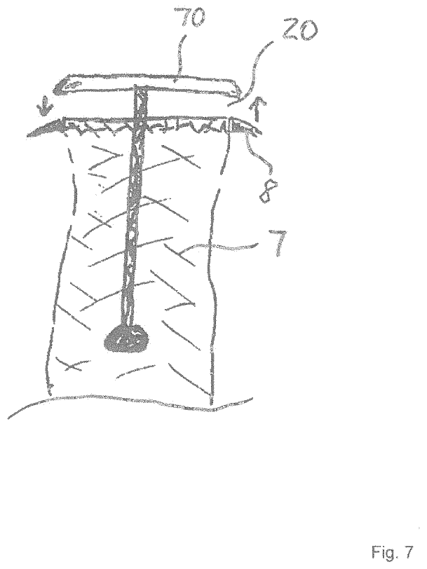

FIG. 7 shows a diagrammatic depiction of a closing device with a lifting cover;

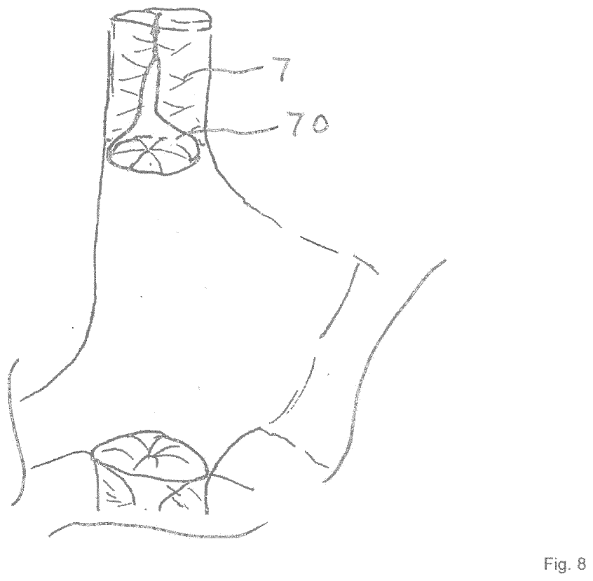

FIG. 8 shows a diagrammatic depiction of a closing device in a vein with a lifting hopper;

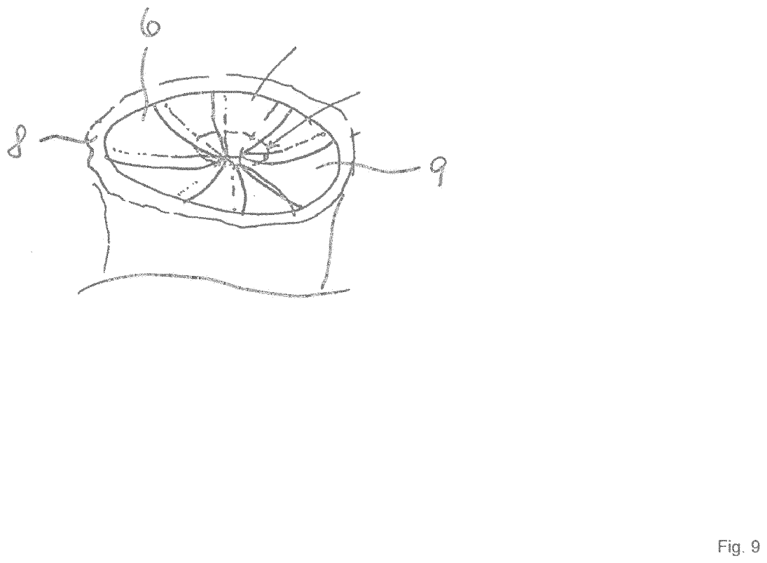

FIG. 9 shows a diagrammatic depiction of a different valve device; and

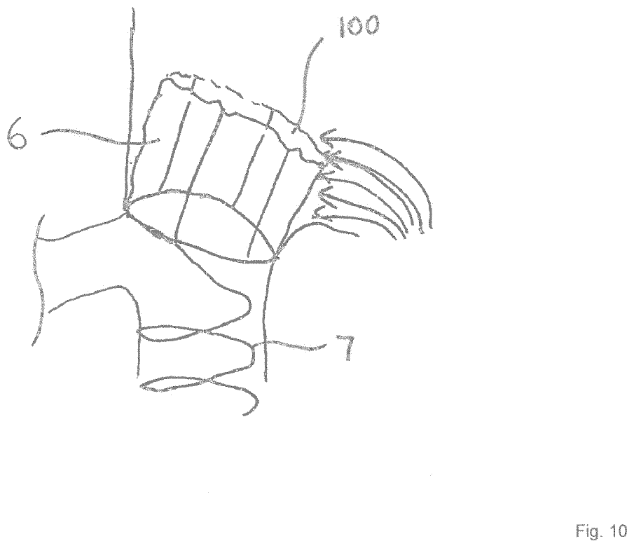

FIG. 10 shows a diagrammatic depiction of another valve device.

FIG. 1 shows a diagrammatic depiction of a minimally-invasive implantable closing device 1 in the area of a vein 2. By means of the closing device 1, the flow into the vein 2 can be opened and closed, wherein a flow is made possible only in the direction of flow that is shown in FIG. 1 by means of arrow A. In the opposite direction, the closing device 1 closes and prevents any backflow.

A section of a vena cava 3, a section of a hepatic vein 4, as well as a section of the right chamber of the heart 5 are shown.

The closing device 1 has a valve device 6, an anchoring device 7, as well as a flexible sealing ring 8, which is formed in a circumferential manner. In the embodiment that is shown, the anchoring device 7 is designed as a self-expanding anchor that has a spiral 7a.

The valve device 6 is designed with closing elements 9 that can be moved and arranged or stretched around a central mast 10. Flaps that are finlike and arranged laterally overlapping are formed with the closing elements 9, as can be seen in greater detail from FIG. 2. In order to make a flow possible, the closing elements 9 tilt or pivot (cf. position with broken lines in FIG. 2), so that a passage 20 between adjacent closing elements is made possible.

FIG. 3 shows a diagrammatic depiction of elements of another embodiment of the closing device 1. The closing elements 9 are opened when flow takes place from below, so that the flow space 20 is opened up. In an upper area 30, the closing elements 9 are edged by a seam 31.

FIG. 4 shows a diagrammatic depiction of a different embodiment of the closing device 1, which is implanted in a minimally-invasive manner. The valve device 6 is formed with the closing elements 9, which have two valves 40, 41, which in each case can be pivoted around a pivoting axis 42. The anchoring device 7 is formed with a stent 43, on which barbs 44 are provided in order to support the anchor. As an alternative, it is shown in addition in FIG. 4 that the closing elements 9 can also pivot on one another (against one another) in order to open the valve device 6.

FIG. 5 shows a diagrammatic depiction of elements of an embodiment of the closing device 1, in which the closing elements 9 of the valve device 6 have an individual pivotable valve 50. The closing element 9 is sewn in a nitinol ring 51.

FIG. 6 shows diagrammatic depictions of another embodiment in which the closing elements 9 of the closing device 1 are formed by means of lifting disks 60, which lift from below with the upstream flow in order to open up the flow space 20 between adjacent closing elements. Viewed from above, adjacent closing elements overlap on the edge side.

FIG. 7 shows a diagrammatic depiction of a different embodiment of the closing device, in which the closing element is formed by means of a lifting cover 70, which lifts from below when flow arrives and opens up a flow space 20.

With comparable functionality, the embodiment of the closing device is shown in FIG. 8, in which the lifting cover 60 is made pear-shaped.

In the embodiment in FIG. 9 of the closing device, the closing elements 9 lie in comparably overlapping petals in the closed state inside the flexible sealing ring 8. When the arrangement of closing elements 9 receives flow, the closing elements 9 pivot and open up the flow area between adjacent closing elements.

In another embodiment, the valve device 6 of the closing device 1 is formed with a hose 100 that forms a closing element 9 and expands when flow arrives, and the opening then pulls together again in a closing manner.

The features disclosed in the description above, the claims, as well as the drawing can be important both individually and in any combination for the implementation of the various embodiments.

1.-16. (canceled)

17. Arrangement of a minimally-invasive implantable closing device (1) in the superior or inferior vena cava of a human body, with:
a valve device (6), in which closing elements (9), which in each case extend flat over a joint surface, and can be moved between a closing position, in which the closing elements (9) together close a valve opening, and an opening position, in which a flow is released through the valve opening, wherein some or all of the closing elements (9) are designed as surface-rigid closing elements;
an anchoring device (7) that has a self-expandable anchor and is set up to anchor the valve device (6) in the area of the superior or inferior vena cava adjacent to the vein opening in the right chamber of the heart; and
a flexible packing collar (8).

18. Arrangement according to claim 17, characterized in that adjacent closing elements overlap in edge-side sections at least in the closing position.

19. Arrangement according to claim 17, characterized in that the valve device (3) is formed as a one-way valve.

20. Arrangement according to claim 17, characterized in that some or all of the closing elements (9) are correspondingly arranged in the closing position of a closing surface of a cone peripheral surface.

21. Arrangement according to claim 17, characterized in that some or all of the closing elements (9) are arranged in the closing position to form a closing surface that is arranged crosswise to a direction of flow.

22. Arrangement according to claim 21, characterized in that the closing elements (9) can be moved some distance apart to move from the closing position into the opening position by means of lifting in the direction of flow.

23. Arrangement according to claim 17, characterized in that the closing elements (9) can be moved by means of pivoting between the closing position and the opening position.

24. Arrangement according to claim 23, characterized in that the closing elements (9) can pivot around a pivoting axis, which elements are arranged in the area of a plane that is stretched from the flexible packing collar (8) or adjacent thereto.

25. Arrangement according to claim 23, characterized in that the pivoting axis is formed to intersect the valve opening.

26. Arrangement according to claim 17, characterized in that some or all of the closing elements (9) are connected at least in pairs by means of holding elements.

27. Arrangement according to claim 17, characterized in that the anchoring device (7) has at least one of the following anchoring sections:
a first anchoring section, which is covered with pericardium or a synthetic membranous plastic material, and
a second anchoring section, which is free of pericardium and the synthetic membranous plastic material.

28. Arrangement according to claim 17, characterized in that the self-expandable anchor has at least one anchor from the following group: self-expandable spiral element and self-expandable stent.

29. Arrangement according to claim 17, characterized in that some or all of the closing elements consist of a biological material.

30. Arrangement according to claim 17, characterized in that some or all of the closing elements consist of a non-biological material.

31. Minimally-invasive implantable tricuspid valve prosthesis, with an arrangement according to claim 17.

32. Minimally-invasive implantable tricuspid valve prosthesis adapted to be arranged according to claim 17.
